(19) United States
(12) Patent Application Publication
(10) Pub. No.: US 2025/0378964 A1
(43) Pub. Date: Dec. 11, 2025

(54) PREDICTING EJECTION FRACTION FROM ECHOCARDIOGRAM VIDEOS VIA A VIDEO VISION TRANSFORMER

(71) Applicant: BOARD OF SUPERVISORS OF LOUISIANA STATE UNIVERSITY AND AGRICULTURAL AND MECHANICAL COLLEGE, Baton Rouge, LA (US)

(72) Inventors: Mohammad Alfrad Nobel Bhuiyan, Shreveport, LA (US); Christopher Kevil, Shreveport, LA (US); Steven A. Conrad, Shreveport, LA (US); Taymaz Akan, Shreveport, LA (US)

(21) Appl. No.: 19/229,923

(22) Filed: Jun. 5, 2025

Related U.S. Application Data
(60) Provisional application No. 63/656,564, filed on Jun. 5, 2024.

(51) Int. Cl. G16H 50/30; G06T 7/00; G16H 30/20

(52) U.S. Cl. CPC G16H 50/30; G06T 7/0016; G16H 30/20; G06T 2207/10016; G06T 2207/10132; G06T 2207/20081; G06T 2207/20084; G06T 2207/30048

(57) ABSTRACT

A method comprising is described herein comprising receiving first magnetic resonance imaging (MRI) data of a first plurality of subjects, wherein the first MRI image data comprises a first plurality of two-dimensional (2D) images, pre-processing the first MRI data for analysis, converting each two-dimensional image of the first plurality of 2D images into first tokens as input for a transformer encoder, wherein the transformer encoder comprises a time series classification transformer, training the transformer encoder using the first input tokens, receiving second MRI data of a subject, wherein the second MRI data comprises a second plurality of 2D images, converting each image of the second plurality of 2D images into second tokens as input to the trained transformer encoder, and applying the trained transformer encoder to the second tokens to predict a state of disease in a subject.

### RELATED APPLICATION

This application claims the benefit of U.S. Application No. 63/656,564, filed Jun. 5, 2024.

### TECHNICAL FIELD

The present disclosure relates to systems and methods that combine driving and health assessments to determine driving risk.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BACKGROUND

Heart disease is the leading cause of death worldwide, and cardiac function as measured by ejection fraction (EF) is an important determinant of outcomes, making accurate measurement a critical parameter in pt evaluation. Echocardiograms are commonly used for measuring EF, but human interpretation has limitations in terms of intra, and inter-observer (or reader) variance. Deep learning (DL) has driven a resurgence in machine learning, leading to advancements in medical applications. We introduce the ViViEchoformer DL approach, which uses a video vision transformer to directly regress the left ventricular function (LVEF) from echocardiogram videos. The model accurately captures spatial information and preserves inter-frame relationships by extracting spatiotemporal tokens from video input, allowing for accurate, fully automatic EF predictions that aid human assessment and analysis. The ViViEchoformer's prediction of ejection fraction has a mean absolute error of 6.14%, root mean squared error of 8.4%, mean squared log error of 0.04, and an R\hat{ }2 of 0.55. Vi ViEchoformer predicted cardiomyopathy with an area under the curve of 0.83 and a classification accuracy of 87 using a standard threshold of less than 50% ejection fraction. Our video-based method provides precise left ventricular function quantification, offering a reliable alternative to human evaluation and establishing a fundamental basis for echocardiogram interpretation.

### BRIEF DESCRIPTION OF THE FIGURES

The file of this patent or application contains at least one drawing executed in color. Copies of this patent or patent application publication with color drawing(s) will be provided to the Office upon request and payment of the necessary fee.

FIG. 1 shows the model pipeline for video regression, under an embodiment.

FIG. 2 shows a graph that illustrates the model's loss over epochs for training (blue) and validation (orange) datasets, under an embodiment.

FIG. 3A-3F illustrate Model Evaluation, under an embodiment.

FIG. 4A-4B illustrate classification accuracy of 85 and AUC of 0.83 using a standard threshold of less than 50% EF. FIG. 4A shows a ROC curve. FIG. 4B shows a confusion matrix where HFrEF, labeled as 0 and Non-HFrEF labeled 1, under an embodiment.

FIG. 5 shows a scatter plot of true vs. predicted LVEF values by the regression model, illustrating classification accuracy relative to the 50% threshold, under an embodiment.

FIG. 6A-6D show comparative distribution of EF values in the training dataset before and after data augmentation and down-sampling techniques, under an embodiment.

FIG. 7 shows sequential visualization of echocardiogram frame preprocessing, under an embodiment.

FIG. 8 shows the layered structure of a neural network model, under an embodiment. The diagram showcases the arrangement of various layers, including multi-head attention, encoder, and dense layers, illustrating the flow of information within the model.

### DETAILED DESCRIPTION

### Introduction

Cardiovascular diseases (CVDs) encompass a range of conditions that can negatively impact the health of the cardiovascular system, which consists of the heart and blood vessels. CVDs are consistently ranked as one of the top causes of death worldwide¹. Heart failure (HF) is a rapidly growing cardiovascular condition, with an estimated prevalence of 37.7 million individuals worldwide. HF is a chronic phase of cardiac functional impairment, causing symptoms such as dyspnea, fatigue, poor exercise tolerance, and fluid retention, which impact patients' quality of life and contribute to the global health crisis². It also carries a high mortality rate. Diagnosing heart failure requires an accurate assessment of cardiac function, which can be done using various methodologies to quantify and characterize. Left ventricular EF is one of the most important metrics for assessing cardiac function, which measures how well the left ventricle can eject blood^{3,4}.

Standard methods for estimating left ventricular ejection fraction include echocardiograms, cardiac MRI, cardiac computed tomography (CT), and Equilibrium Radionuclide Angiocardiography (ERNA). Echocardiography uses ultrasound to create real-time images of the heart's chambers, valves, and blood flow, assessing the volume of blood pumped out of the left ventricle with each contraction. MRI provides detailed images of the heart's structure and function but has limitations such as cost, availability, and potential contraindications. CT uses X-rays to produce detailed heart images but has limitations such as radiation exposure, allergic reactions to contrast media, and limited dynamic heart function assessment. Equilibrium radionuclide angiography is a method used in nuclear medicine studies. Still, it has some drawbacks, like taking a long time to process, injecting radiopharmaceutical agents, and yielding low resolution for regional ventricular function in heart disease patients^{3,5}. Clinically echocardiography is the preferred most common method for estimating LVEF because it is widely available, provides real-time imaging, is non-invasive, and is more cost-effective than other options. This makes it particularly useful for quick and detailed assessments in various clinical situations.

Traditional echocardiography typically includes a visual interpretation to estimate LVEF, providing a qualitative assessment without precise numerical values. This approach is well-suited for managing acute patients but falls short when it comes to serial evaluations, particularly in patients with valvular lesions causing regurgitation. There are also quantitative capabilities for echocardiography using the Simpson's method and fractional shortening to calculate EF. The human calculation of ejection fraction is subject to variability due to irregular heart rate and the nature of the calculation, which necessitates manual ventricle size tracing for every beat⁴. The variability in estimating LVEF among different observers can often result in requests for additional testing, review of the study, and reinterpretation, which can impact the timing of therapeutic interventions⁵⁻⁷.

Conventional Machine learning (ML) has recently led to substantial advancements in diverse fields, including medical applications. Conventional ML has been utilized in echocardiography to determine the ejection fraction, with significant interest in their potential to provide improvement in disease diagnoses, aid decision-making, and serve as a confirmatory assessment^{8,9}. However, conventional ML has a potential disadvantage in its reliance on feature engineering, which is a manual and time-consuming process. Moreover, despite obtaining images in various positions and orientations, these conventional echocardiographic systems lack 3D localization and spatial relation measurements for volume computation.

Deep learning has driven a significant resurgence in machine learning due to availability of large data sets, and advances in computing power¹⁰⁻¹⁵. This field has revolutionized machine learning by understanding and manipulating data, including images^{16,17}, and incorporation of natural language processing (NLP)¹⁸. Moreover, deep learning differs from conventional methods as it avoids manual feature engineering. Also, using deep learning techniques in medical diagnostics improves the accuracy of diagnoses^{19,20}. It plays a crucial role in predictive analytics, allowing for detecting possible health risks or outcomes²¹. These techniques provide healthcare professionals with valuable predictive insights through the assimilation and analysis of various datasets, including patient information, genetic profiles, imaging studies, and clinical records, and enables early detection of diseases or health deterioration²².

Deep learning techniques can be used to determine ejection fraction, estimate end-diastole and end-systole volumes, and calculate the percentage difference between them, rather than relying on actual echocardiogram videos²³⁻²⁷. A recently proposed method, EchoNet-Dynamic⁴, directly regresses LVEF from video inputs using spatiotemporal models, which avoids the need to estimate EDV and ESV separately. EchoNet-Dynamic, a video-based deep learning model, has been proposed for echocardiograms, demonstrating its ability to assess ejection fraction accurately across the entire video. It is a CNN model that uses atrous convolution²⁸ for semantic segmentation of the left ventricle, a CNN model²⁹ with residual connections and spatiotemporal convolutions for predicting the ejection fraction, and video-level predictions for beat-to-beat estimations of cardiac function. Moreover, another video-based method performs LV segmentation using echocardiogram sequences and then converts the predicted context into an end-to-end video regression model³⁰. However, segmentation, a sensitive process involving categorizing entire regions, may increase computational requirements and processing times. Inaccuracies in segmentation can impact subsequent classification or regression tasks, making the overall process more sensitive to segmentation quality. Recent advances in deep learning have shown that it can accurately and reproducibly identify human-identifiable phenotypes and characteristics not recognized by human experts, overcoming limitations in human interpretation³¹⁻³³.

Herein, we propose an end-to-end deep learning approach, ViViEchoformer, which leverages a video vision transformer (ViViT)³⁴ to regress LVEF from echocardiogram videos directly. We converted ViViT from classification to regression to predict LVEF. The model captures spatial information and preserves inter-frame relationships by extracting spatiotemporal tokens from the input video. While utilizing the video vision transformer to capture spatiotemporal patterns in the video accurately, this method performs precise, fully automatic EF predictions that facilitate human assessment and subsequent analysis.

TABLE 1
Details of model variants

| | |
|---|---|
| Parameter name | Values |

Hyperparameters of ViViT

| | |
|---|---|
| Optimizer | SGD |
| Batch size | 128 |
| Epoch | 100 |
| Input Shape | (52, 52, 32, 1) |
| Layer norm | 1e−6 |
| Learning rate | 1e−4 |
| Number of heads | 12 |
| Number of Layers | 10 |
| Patch size | (32, 8, 8) |
| Projection dim | 512 |

### Results

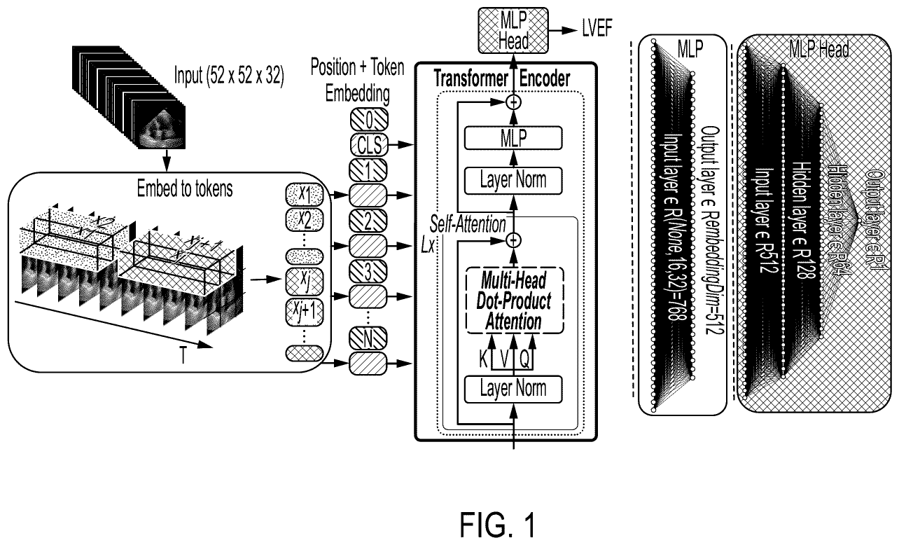

Our neural network architecture was implemented in Python using the TensorFlow and Keras libraries. A workstation equipped with 62 GB of RAM and an NVIDIA Geforce GTX 4080 GPU was used for all experiments. We trained our transformer model (FIG. 1) on a data set with 10,030 echocardiogram videos provided by Stanford University Hospital³⁵. We converted the classification model ViViT, into a regression model and trained it to estimate the left ventricular ejection fraction from echocardiogram videos using a training and validation set of over 30700 and 1200 videos, respectively, and a test set of over 1200 videos. Note that the reference Arnab, A. et al. ViViT: A Video Vision Transformer. 6836-6846 Preprint at (2021) is incorporated herein by reference in its entirety.

To convert ViViT into a regression model, we replaced the final classification head with a multi-layer perceptron (MLP) that outputs a single continuous value (e.g., LVEF). The model is trained using mean squared error (MSE) loss to learn the mapping from spatiotemporal video features to a continuous clinical outcome. The video vision transformer model shown in FIG. 1 below comprises a multi-head attention layer and a feed forward neural network layer. The output of the multi-head attention layer is fed into the feed forward neural network layer. The output of the feed forward neural network is fed into the multilayer perceptron head.

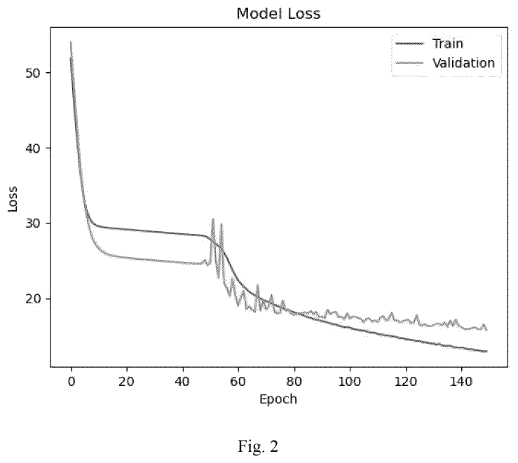

The analysis focused on the 32 frames of videos that were resized to 52×52 dimensions. We employed the SGD optimizer for training, and the training process was conducted over 100 epochs with a batch size of 128 and a learning rate of 1e-4. Table 1 provides a summary of the configuration of the training parameters. The model checkpoint is configured to save only the optimal solution discovered during training based on the loss function evaluation during validation. The model checkpoint is saved when a metric improves on a validation set during training. As depicted in FIG. 2, the model demonstrates a significant reduction in loss in the initial epochs, which indicates the model's capacity to learn quickly from the training data.

FIG. 1 shows the model pipeline for video regression. The Tubelet embedding technique extracts and linearly embeds nonoverlapping tubelets across the spatio-temporal input volume. Using spatial-temporal attention, the transformer encoder forwards all spatio-temporal tokens extracted from the video.

FIG. 2. The graph illustrates the model's loss over epochs for training (blue) and validation (orange) datasets.

We have employed the evaluation metrics for evaluating the performance of ViViEchoformer on the EchoNet test dataset, which were not previously used during model training. The estimation of EF has been associated with interobserver variability of up to 14%³⁶. The ViViEchoformer's prediction of ejection fraction had a mean absolute error of 6.14%, root mean squared error of 8.4%, mean squared log error of 0.03, and an R² of 0.55.

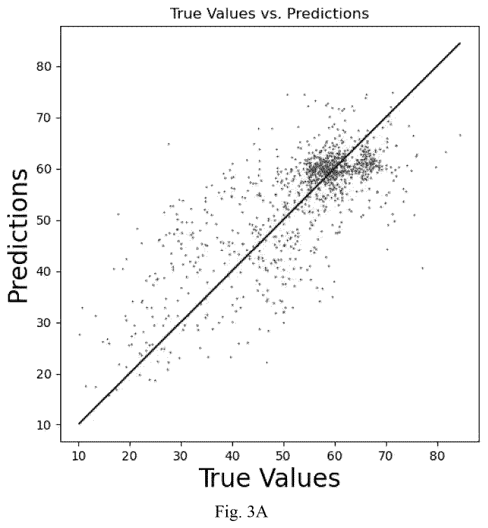

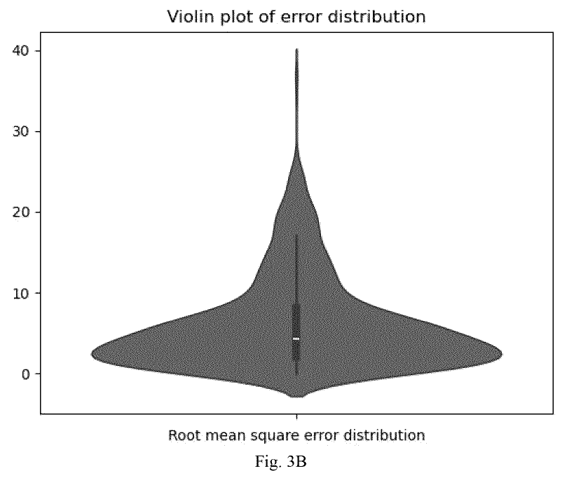

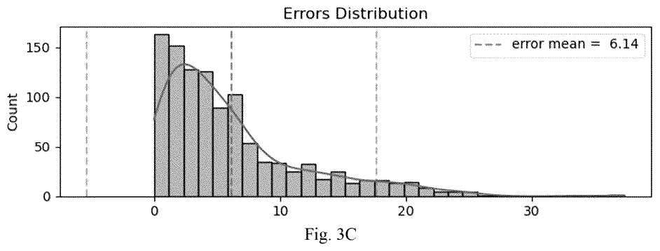

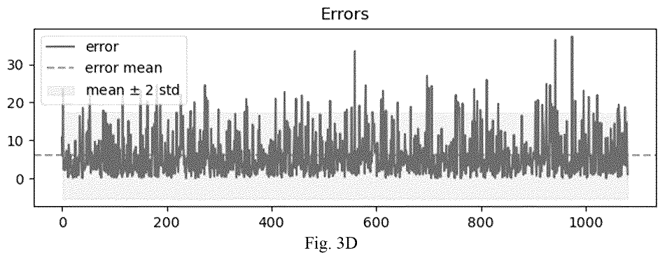

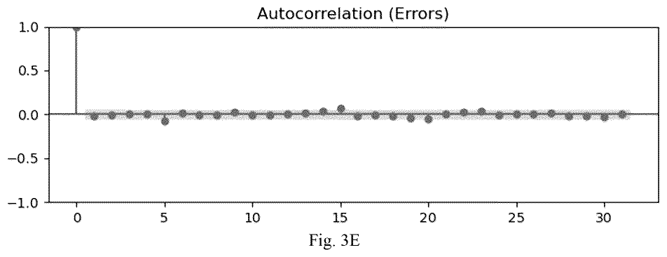

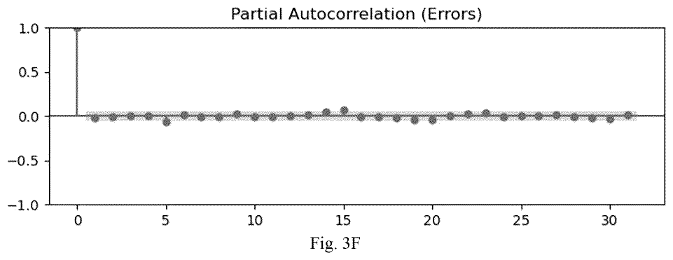

The visual assessment has been carried out using six plots (FIG. 3). These plots are used to evaluate the performance of a predictive model, providing information about the accuracy, distribution of errors, and independence of errors, which are crucial for validating the robustness of the model. The scatter plot of FIG. 3A shows the model's predictions against the actual values, with points scattered around the line of perfect agreement. This indicates that the model captures the trend in the data, but the spread of points away from the line indicates variances in prediction accuracy. The violin plot and histogram of error distribution FIG. 3B and FIG. 3C provide insight into the distribution of prediction errors, with a long tail of errors indicating a right-skewed distribution. The line plot of errors in FIG. 3D shows variability, with most falling within two standard deviations of the mean. However, occasional spikes beyond this range suggest more significant errors, possibly due to outliers or less valid assumptions. The autocorrelation and partial autocorrelation plots in FIGS. 3E and 3F show that the errors are mostly independent, indicating a positive predictive model performance.

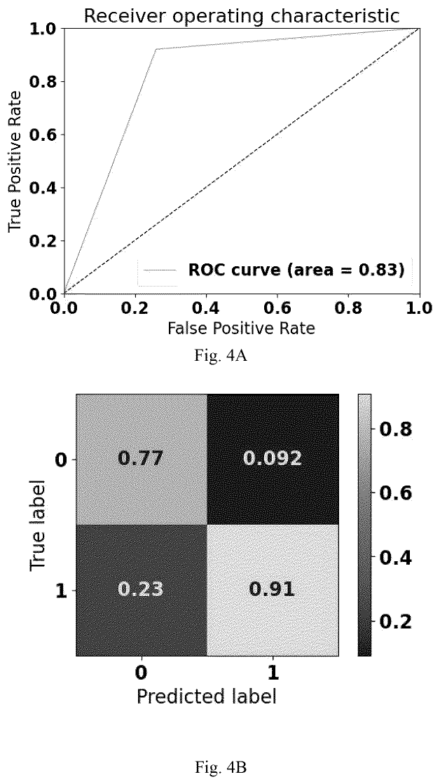

Table 2 reports the model's classification performance distinguishing between Heart Failure with Reduced Ejection Fraction (HFrEF) and Non-HFrEF cases. Precision, recall, f1-score, and support numbers are reported for both categories. The classification report shows ViViEchoformer's prediction of cardiomyopathy with an area under the curve of 0.83 (FIG. 4A), using a common threshold of an EF of less than 50%. The model achieves a precision of 0.77 for HFrEF cases, indicating 77% correctness, and a recall of 0.83, indicating 83% correct identification. The f1-score balances these metrics, indicating the model's effectiveness in HFrEF cases. However, the model performs better for non-HFrEF cases, with a precision of 0.91 and recall of 0.92, resulting in a higher f1-score of 0.89. The overall accuracy across both classes is 0.87, indicating 87% correct classifications. The macro average f1-score is 0.83, considering the balance between classes without weighting for their representation in the dataset. The weighted average f1-score is also 0.87, indicating consistent high performance across classes when accounting for the number of samples in each. FIG. 4B illustrates the confusion matrix for our model's classification performance where HFrEF is labeled 0, and Non-HFrEF is labeled 1. The matrix visually represents the model's predictions compared to the actual labels.

FIG. 3A shows comparison of ViViEchoformer predicted, and EchoNet dataset reported ejection fractions (n=1288). FIG. 3B shows the violin plot showcasing the model error distribution. FIG. 3C shows an errors distribution histogram. FIG. 3D shows error values across samples. FIG. 3E shows autocorrelation plot of residuals. FIG. 3F shows partial autocorrelation of the residuals.

### Discussion

The most prominent architecture of choice in sequence modeling is the transformer, which uses a multi-headed self-attention mechanism instead of convolution. ViViEchoformer is a video transformer-based deep learning model for echocardiogram video understanding tasks, allowing for accurate, fully automatic EF predictions that aid human assessment and analysis. To our knowledge, ViViEchoformer is the first deep-learning model that uses transformers to estimate the ejection fraction from echocardiogram videos. Previous attempts to use deep learning techniques are primarily used to determine EFs, end-diastole and end-systole volumes, and percentage differences in echocardiogram videos rather than actual data. These methods typically do not account for inter-frame relationships or temporal dependencies within the video sequences during their analysis. To process video sequences, ViViEchoformer splits them up into smaller temporal and spatial units known as tokens. The model can then comprehend temporal dependencies throughout the sequence and spatial relationships within individual frames thanks to extracting and processing information from these tokens across frames.

TABLE 2
Classification performance for HFrEF and Non-HFrEF cases

| | precision | recall | f1-score | support |
|---|---|---|---|---|
| HFrEF | 0.77 | 0.74 | 0.75 | 285 |
| Non-HFrEF | 0.91 | 0.92 | 0.71 | 794 |
| accuracy | | | 0.87 | 1079 |
| macro avg | 0.84 | 0.83 | 0.83 | 1079 |
| weighted avg | 0.87 | 0.87 | 0.87 | 1079 |

FIGS. 4A-4B shows classification accuracy of 85 and AUC of 0.83 using a standard threshold of less than 50% EF. FIG. 4A shows an ROC curve. FIG. 4B shows a confusion matrix where HFrEF, labeled as 0 and Non-HFrEF labeled 1.

Some video-based methods perform LV segmentation using echocardiogram sequences, but segmentation may increase computational requirements and processing times due to its sensitive nature. However, when analyzing massive datasets, DL techniques can reveal hidden patterns that were previously not apparent. Recent advancements in DL techniques have demonstrated their ability to “see the unseen” in images and videos. Consequentially, determining EFs without end-diastole and end-systole volumes could be possible for DL techniques. Without infusing knowledge awareness and using any pre-processing, such as segmentation, our method directly regresses EF among the video frames. ViViEchoformer's predictions have a variance comparable to or less than human experts' measurements of cardiac function³⁷. ViViEchoformer achieved high prediction accuracy for estimating ejection fraction performed by human interpreters. Its prediction of ejection fraction had a mean absolute error of 6.14%, which is within the typical inter-observer variation of 14%.

In the study by Ouyang et al.⁴, five expert sonographers and cardiologists conducted a blinded review of echocardiogram videos that exhibited the largest absolute differences between the initial human labels and the predictions made by EchoNet-Dynamic. These experts independently assessed the relevant videos and two blinded measurements of ejection fraction. The findings revealed that 38% (15 out of 40) of the videos had significant issues related to video quality or the acquisition process. In comparison, 13% (5 out of 40) were characterized by marked arrhythmias, which constrained the experts' capacity to assess ejection fraction accurately. A critical limitation of the EchoNet-Dynamic dataset stems from the inaccuracy in the initial human labeling of echocardiogram videos, compounded by issues related to poor image quality, arrhythmias, and variations in heart rate. These factors significantly impact the training and evaluation performance of our model.

In developing a model to regress the left ventricular ejection fraction (LVEF) from echocardiogram videos, we encountered a nuanced issue at the intersection of statistical significance and clinical utility, particularly when classifying LVEF based on the 50% cutoff. Our model is capable of closely approximating actual LVEF values. Yet, we observe instances where minor discrepancies—such as a predicted LVEF of 49.9% versus an actual measurement of 50.01%—raise important considerations. While these small differences may be statistically significant, they highlight the clinical uncertainty of near-threshold predictions in model evaluation. This distinction is important because, in clinical practice, the marginal difference may not change treatment or patient outcome, calling statistically significant but clinically marginal model predictions into question. This is a limitation for most methodologies, and should be acknowledged.

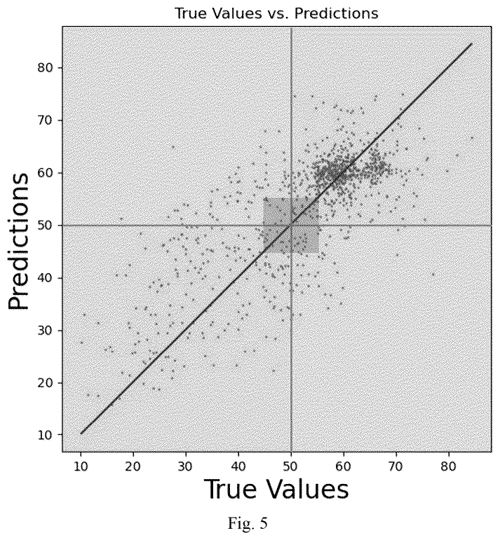

FIG. 5 shows a scatter plot of true vs. predicted LVEF values by the regression model, illustrating classification accuracy relative to the 50% threshold. The green area represents correct classifications, while the orange area signifies incorrect classifications. The highlighted square around the 50% line delineates the zone of uncertainty, emphasizing the model's challenge in making near-threshold predictions.

FIG. 5 presents a scatter plot evaluating the performance of a regression model that predicts left ventricular ejection fraction (LVEF). The true LVEF values are on the X-axis, while the Y-axis displays the model's predicted LVEF values. The overlay of a green zone and an orange area indicates the boundary of correct and incorrect classifications by the model relative to the critical threshold of 50%. The green zone indicates regions where the model's predictions align correctly with the true classifications—predictions of LVEF less than 50% that are indeed below 50% (lower left) and predictions above 50% that are actually above 50% (upper right). Conversely, the orange zone indicates regions of misclassification—predictions above 50% for true values below 50% (lower right) and vice versa (upper left). Central to the plot is a highlighted square around the 50% line, visually representing the area of uncertainty where the model's predictions are close to the threshold, encapsulating the challenge of near-threshold predictions. This zone of uncertainty underscores the difficulty in achieving precise classifications around the 50% cutoff point, which is critical for clinical decision-making based on LVEF values.

The study suggests that future research could focus on developing advanced classification models to identify videos with poor image quality, arrhythmias, and heart rate variations. This would improve the reliability of automated assessments by reducing the impact of the issues mentioned earlier on model predictions, thereby enhancing the accuracy of ejection fraction prediction.

### Method

### Data Management

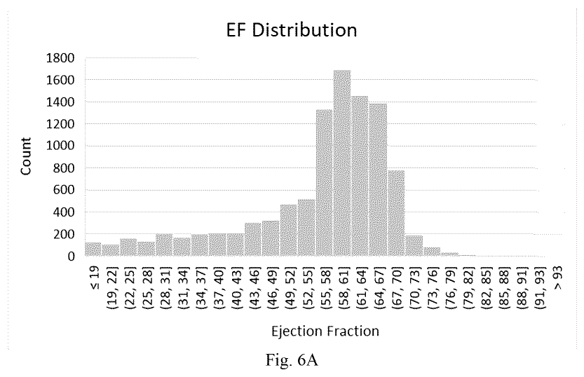

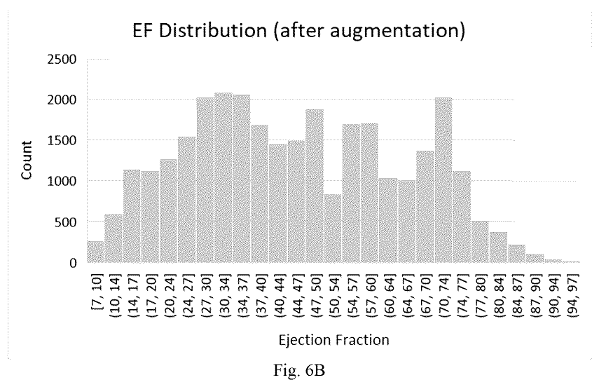

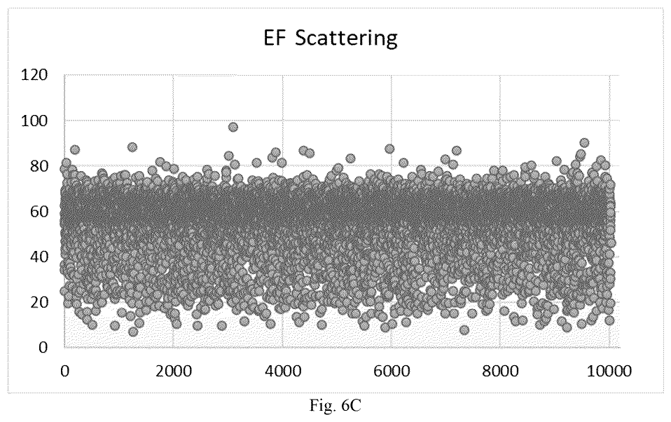

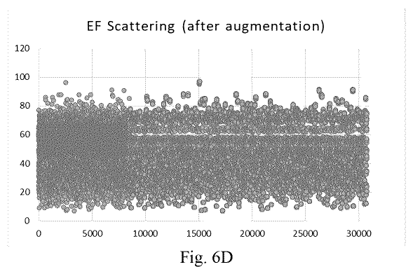

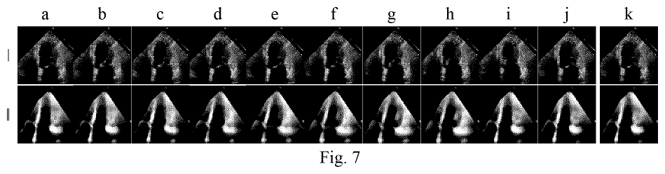

The study used a dataset of 10,030 apical-4-chamber echocardiography videos from patients at Stanford University Hospital between 2016 and 2018³⁸. The data was meticulously preprocessed to ensure integrity and uniformity, including cropping and masking operations. The videos were then down-sampled to a uniform resolution of 112×112 pixels using cubic interpolation, ensuring the quality of the visual data and compatibility with the analytical framework. This dataset is crucial for understanding cardiac function representations in full resting echocardiogram studies. The dataset was divided into test, validation, and training sets, with 1,277, 1,288, and 7,462 videos in each set. The histogram in FIG. 6A visually represents the EF values in the training set, showcasing the range from 6.90 to 96.96. The histogram shows a dataset's imbalanced distribution of ejection fraction values, with a significant concentration in the 55% to 70% range. Consequently, the pattern and spread of EFs around the line indicate how the points in the 55% and 70% ranges are closely scattered around a diagonal line (FIG. 3A). This imbalance can affect the performance of predictive models trained on this data, potentially leading to bias toward predicting values in the most common range. Additionally, a scatter plot was included to illustrate the spread of ejection fraction values within the training dataset (FIG. 6C and FIG. 6D).

In the initial examination of our training dataset, we identified a skewed distribution of EF values, which threatened to bias our predictive model towards the more common EF ranges, thereby impairing its generalizability. We first addressed the variability in frame counts to ensure uniformity in video clip length. Videos with fewer than 32 frames were lengthened by padding the last frame, whereas for videos with fewer than 64 frames, we employed 32 random samples to standardize their length. For videos containing 64 frames or more, we generated 32-frame echocardiogram clips by sampling every second frame. This preprocessing protocol was applied to all videos to create a consistent structure for subsequent steps. Following this standardization, we specifically targeted the underrepresented EF values for augmentation. For videos with an excess of 64 frames, we generated two distinct clips with variable starting points by sampling every other frame, effectively doubling the representation of these EF ranges. This augmentation, performed prior to any down-sampling, was crucial in addressing the initial data imbalance. In the subsequent phase, we down-sampled the overrepresented EF values to balance the dataset. Later, the underrepresented values are applied to each frame through a series of image transformations, including rotation, zoom, shift, and shear. A random factor between 0.99 and 1.01 also changed the EF value for each augmented video. This was done to maintain physiological plausibility and add a realistic range. The histogram in FIG. 6B visually represents the EF values in the training set after augmentation.

### Preprocessing

Accurately assessing cardiac function using echocardiograms is crucial to minimize noise and ensure high-quality data for accurate interpretation. To address this, we developed a comprehensive preprocessing pipeline that enhances the interpretability of echocardiogram frames.

FIGS. 6A-6D show a comparative distribution of EF values in the training dataset before and after data augmentation and down-sampling techniques. The initial dataset (FIG. 6A) consisted of 7462 samples, while the augmented dataset (FIG. 6B) expanded to 30787 samples, illustrating the effect of augmentation and balancing strategies on the EF value distribution. FIG. 6C and FIG. 6D represent the spread of EF before and after augmentation in the training dataset.

The preprocessing method starts with 32 echocardiogram frames with a 52×52 pixel resolution. The median frame is calculated by determining the median value of each pixel location across all 32 frames (temporal dimension), resulting in a singular 52×52 matrix. Then, a frame-wise multiplication operation is performed on each original video frame, resulting in a transformed video with identical dimensions but modified pixel values by multiplying the corresponding median pixel values. This meticulous operation is performed for all 32 frames in the sequence. Subsequently, histogram equalization was applied to each frame to adjust contrast and improve the visibility of cardiac structures, followed by a median blur filter with a 3×3 pixel mask to reduce noise while preserving essential anatomical details. FIG. 7 compares the first 10 frames of the original video and their preprocessed counterparts, showing the significant improvements.

FIG. 7 shows a sequential visualization of echocardiogram frame preprocessing. The top row (a-k) displays the first 11 original frames from the echocardiogram video, demonstrating the raw imaging data. After applying our preprocessing steps, the bottom row (a-k) illustrates the corresponding frames: median frame calculation, frame-wise multiplication, histogram equalization, and median blur filtering. The processed frames reveal a marked enhancement in the definition and contrast of cardiac structures, providing a clear visual distinction from the original frames and underscoring the efficacy of the preprocessing technique.

### Video-Based Transformer Model and Training

The Vision Transformer (ViT) is a pure-transformer architecture that has outperformed convolutional neural networks in image classification, offering a competitive alternative to the widely used convolutional neural networks in computer vision^{34,39}. The ViViT architecture, inspired by the ViT, provides a new approach to video classification. It uses transformer-based models, leveraging attention-based mechanisms to model long-range contextual relationships in video content. This innovative approach offers a strategic alternative to conventional 3D CNNs⁴⁰ and RNNs⁴¹, allowing for more accurate and efficient video classification.

Even though ViViT is an efficient video classification model, we trained the ViViT from scratch to directly regress the LVEF from echocardiogram videos. The model performed self-attention, computed on a sequence of spatio-temporal patches we extracted from the echocardiogram videos. We replaced the final layer of the classifier head, intended to output various classes, with a new layer designed to produce a single, continuous output. There is only one output unit in this new layer and no activation function. The tubelet embedding of the echocardiogram frames feeds the model with nonoverlapping spatiotemporal information. The tubelet embedding extracts non-overlapping spatiotemporal patches from the video frames, providing the model with localized spatiotemporal information as input. Here, “Tubelet embedding” refers to dividing a video into 3D patches (tubelets) across space and time.

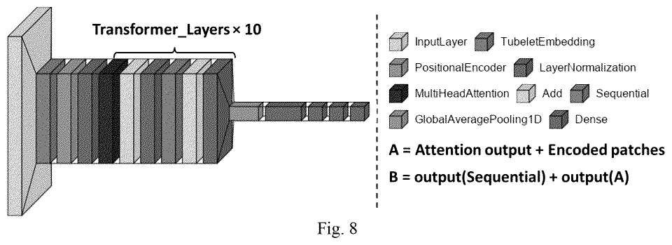

FIG. 8 shows the layered structure of a neural network model. The diagram showcases the arrangement of various layers, including multi-head attention, encoder, and dense layers, illustrating the flow of information within the model.

The model is structured around a sequence of ten transformer layers. Each layer consists of twelve heads. The token size (model dimension) was set to d=512. The hidden size of multi-layer perceptron (MLP) was 768. The output of the tokens is then transformed into a regression prediction via an MLP as non-linearity in the three hidden layers of 512, 128, and 64. FIG. 8 illustrates the layered structure of our model. (Note that FIG. 8 provides a schematic overview of data flow as it passes through the model specified in FIG. 1 beginning with spatio-temporal patches extracted from echocardiogram video and ending in ejection fraction prediction).

### REFERENCES

1. Robinson, S. Cardiovascular disease. Priorities for Health Promotion and Public Health 355-393 (2021) doi: 10.4324/9780367823689-16.
2. Ziaeian, B. & Fonarow, G. C. Epidemiology and aetiology of heart failure. Nature Reviews Cardiology 2016 13:6 13, 368-378 (2016).
3. Savarese, G., Stolfo, D., Sinagra, G. & Lund, L. H. Heart failure with mid-range or mildly reduced ejection fraction. Nature Reviews Cardiology 2021 19:2 19, 100-116 (2021).
4. Ouyang, D. et al. Video-based AI for beat-to-beat assessment of cardiac function. Nature 2020 580:7802 580, 252-256 (2020).
5. Gopal, A. S. et al. Assessment of Cardiac Function by Three-dimensional Echocardiography Compared With Conventional Noninvasive Methods. Circulation 92, 842-853 (1995).
6. Lang, R. M. et al. Recommendations for Cardiac Chamber Quantification by Echocardiography in Adults: An Update from the American Society of Echocardiography and the European Association of Cardiovascular Imaging. Eur Heart J Cardiovasc Imaging 16, 233-271 (2015).
7. Farsalinos, K. E. et al. Head-to-Head Comparison of Global Longitudinal Strain Measurements among Nine Different Vendors: The EACVI/ASE Inter-Vendor Comparison Study. Journal of the American Society of Echocardiography 28, 1171-1181.e2 (2015).
8. Cannesson, M. et al. A Novel Two-Dimensional Echocardiographic Image Analysis System Using Artificial Intelligence-Learned Pattern Recognition for Rapid Automated Ejection Fraction. J Am Coll Cardiol 49, 217-226 (2007).
9. Kim, Y. et al. Extraction of left ventricular ejection fraction information from various types of clinical reports. J Biomed Inform 67, 42-48 (2017).
10. Esteva, A. et al. A guide to deep learning in healthcare. Nature Medicine 2019 25:1 25, 24-29 (2019).
11. van der Laak, J., Litjens, G. & Ciompi, F. Deep learning in histopathology: the path to the clinic. Nature Medicine 2021 27:5 27, 775-784 (2021).
12. Wainberg, M., Merico, D., Delong, A. & Frey, B. J. Deep learning in biomedicine. Nature Biotechnology 2018 36:9 36, 829-838 (2018).
13. Moen, E. et al. Deep learning for cellular image analysis. Nature Methods 2019 16:12 16, 1233-1246 (2019).
14. Esteva, A. et al. Deep learning-enabled medical computer vision. npj Digital Medicine 2021 4:14, 1-9 (2021).
15. Aggarwal, R. et al. Diagnostic accuracy of deep learning in medical imaging: a systematic review and meta-analysis. npj Digital Medicine 2021 4:1 4, 1-23 (2021).
16. Lin, T. Y. et al. Microsoft COCO: Common objects in context. Lecture Notes in Computer Science (including subseries Lecture Notes in Artificial Intelligence and Lecture Notes in Bioinformatics) 8693 LNCS, 740-755 (2014).
17. Russakovsky, O. et al. ImageNet Large Scale Visual Recognition Challenge. Int J Comput Vis 115, 211-252 (2015).
18. Hirschberg, J. & Manning, C. D. Advances in natural language processing. Science (1979) 349, 261-266 (2015).
19. Litjens, G. et al. Deep learning as a tool for increased accuracy and efficiency of histopathological diagnosis. Scientific Reports 2016 6:1 6, 1-11 (2016).
20. Aggarwal, R. et al. Diagnostic accuracy of deep learning in medical imaging: a systematic review and meta-analysis. npj Digital Medicine 2021 4:1 4, 1-23 (2021).
21. Odigwe, B. E., Rajeoni, A. B., Odigwe, C. I., Spinale, F. G. & Valafar, H. Application of machine learning for patient response prediction to cardiac resynchronization therapy. Proceedings of the 13th ACM International Conference on Bioinformatics, Computational Biology and Health Informatics, B C B 2022 (2022) doi: 10.1145/3535508.3545513.
22. Rajeoni, B. et al. Automated Measurement of Vascular Calcification in Femoral Endarterectomy Patients Using Deep Learning. Diagnostics 2023, Vol. 13, Page 3363 13, 3363 (2023).
23. Madani, A., Arnaout, R., Mofrad, M. & Arnaout, R. Fast and accurate view classification of echocardiograms using deep learning. npj Digital Medicine 2018 1:1 1, 1-8 (2018).
24. Ghorbani, A. et al. Deep learning interpretation of echocardiograms. npj Digital Medicine 2020 3:1 3, 1-10 (2020).
25. Wei, H. et al. Temporal-Consistent Segmentation of Echocardiography with Co-learning from Appearance and Shape. Lecture Notes in Computer Science (including subseries Lecture Notes in Artificial Intelligence and Lecture Notes in Bioinformatics) 12262 LNCS, 623-632 (2020).
26. Reynaud, H. et al. Ultrasound Video Transformers for Cardiac Ejection Fraction Estimation. Lecture Notes in Computer Science (including subseries Lecture Notes in Artificial Intelligence and Lecture Notes in Bioinformatics) 12906 LNCS, 495-505 (2021).
27. Jafari, M. H., Woudenberg, N. Van, Luong, C., Abolmaesumi, P. & Tsang, T. Deep bayesian image segmentation for a more robust ejection fraction estimation. Proceedings-International Symposium on Biomedical Imaging 2021-April 1264-1268 (2021).
28. Chen, L.-C., Papandreou, G., Schroff, F. & Adam, H. Rethinking Atrous Convolution for Semantic Image Segmentation. (2017).
29 Tran, D. et al. A closer look at spatiotemporal convolutions for action recognition. in Proceedings of the IEEE conference on Computer Vision and Pattern Recognition 6450-6459 (2018).
30. Dai, W., Li, X., Ding, X. & Cheng, K. T. Cyclical Self-Supervision for Semi-Supervised Ejection Fraction Prediction From Echocardiogram Videos. IEEE Trans Med Imaging 42, 1446-1461 (2023).
31. Lang, R. M. et al. Recommendations for Cardiac Chamber Quantification by Echocardiography in Adults: An Update from the American Society of Echocardiography and the European Association of Cardiovascular Imaging. Eur Heart J Cardiovasc Imaging 16, 233-271 (2015).
32. Poplin, R. et al. Prediction of cardiovascular risk factors from retinal fundus photographs via deep learning. Nature Biomedical Engineering 2018 2:3 2, 158-164 (2018).
33. Hughes, J. W. et al. A deep learning-based electrocardiogram risk score for long term cardiovascular death and disease. npj Digital Medicine 2023 6:16, 1-9 (2023).
34. Arnab, A. et al. ViViT: A Video Vision Transformer. 6836-6846 Preprint at (2021).
35. Ouyang, D. et al. Echonet-dynamic: a large new cardiac motion video data resource for medical machine learning. in NeurIPS ML4H Workshop: Vancouver, BC, Canada (2019).
36. Thavendiranathan, P. et al. Improved Interobserver Variability and Accuracy of Echocardiographic Visual Left Ventricular Ejection Fraction Assessment through a Self-Directed Learning Program Using Cardiac Magnetic Resonance Images. Journal of the American Society of Echocardiography 26, 1267-1273 (2013).
37. Lang, R. M. et al. Recommendations for cardiac chamber quantification by echocardiography in adults: an update from the American Society of Echocardiography and the European Association of Cardiovascular Imaging. European Heart Journal-Cardiovascular Imaging 16, 233-271 (2015).
38. Ouyang, D. et al. Echonet-dynamic: a large new cardiac motion video data resource for medical machine learning. in NeurIPS ML4H Workshop: Vancouver, BC, Canada (2019).
39. Ouyang, D. et al. Echonet-dynamic: a large new cardiac motion video data resource for medical machine learning. in NeurIPS ML4H Workshop: Vancouver, BC, Canada (2019).
40. Feichtenhofer, C. X3d: Expanding architectures for efficient video recognition. in Proceedings of the IEEE/CVF conference on computer vision and pattern recognition 203-213 (2020).
41. Bhardwaj, S., Srinivasan, M. & Khapra, M. M. Efficient video classification using fewer frames. in Proceedings of the IEEE/CVF Conference on Computer Vision and Pattern Recognition 354-363 (2019).

1. A method comprising,
receiving first echocardiogram video data of a first plurality of subjects, wherein the first echocardiogram video data comprises a first plurality of two-dimensional (2D) image sequences;
converting each 2D image sequence of the first plurality of 2D image sequences into first tokens as input for a video vision transformer model, wherein the model comprises a final regression layer to predict ejection fraction;
training the video vision transformer model using the first input tokens;
receiving second echocardiogram video data of a subject, wherein the second echocardiogram video data comprises a second 2D image sequence;
converting each image of the second 2D image sequences into second tokens as input to the trained video vision transformer model;
applying the trained video vision transformer model to the second token to predict an ejection fraction of the subject.

2. The method of claim 1, wherein each 2D image of the first plurality of 2D image sequences and the second 2D image sequence comprises a 52×52 pixel frame.

3. The method of claim 2, wherein each 2D image sequence comprises 32 frames.

4. The method of claim 3, wherein the converting comprises tubelet embedding of the first plurality of 2D image sequences and the second 2D image sequences.

5. The method of claim 4, wherein the tubelet embedding extracts non-overlapping spatiotemporal patches across the 2D image sequences.

6. The method of claim 5, wherein each patch comprises an 8×8 pixel frame.

7. The method of claim 6, wherein the converting comprises projecting the non-overlapping spatiotemporal patches as tokens into a vector.

8. The method of claim 1, wherein the video vision transformer model comprises a multi-head attention layer.

9. The method of claim 8, wherein the video vision transformer model comprises a feed forward neural network layer.

10. The method of claim 9, wherein output of the multi-head attention layer is fed into the feed forward neural network layer.

11. The method of claim 10, wherein the final layer of the video vision transformer model comprises a multilayer perceptron head.

12. A method comprising,
receiving clinical echocardiogram video data of a subject, wherein the clinical echocardiogram video data comprises a two-dimensional (2D) image sequence;
converting each image of the 2D image sequence into tokens as input to a trained video vision transformer model, wherein the trained video vision transform model is converted from classification to a regression model by replacing the final layer of a video vision transformer model with a multiplayer perceptron head to regress ejection fraction from the echocardiogram video data;
applying the trained video vision transformer model to the tokens to predict an ejection fraction of the subject.

13. The method of claim 12, wherein the video vision transformer model is trained using first echocardiogram video data of a first plurality of subjects.

14. The method of claim 13, wherein the first echocardiogram video data comprises a first plurality of two-dimensional (2D) image sequences.

15. The method of claim 14, wherein each 2D image sequence of the first plurality of 2D image sequences is converted into first tokens as input for the video vision transformer model.

16. The method of claim 15, wherein the video vision transformer model is trained using the first tokens.

17. The method of claim 16, wherein each 2D image of the 2D image sequence and the first plurality of 2D image sequences comprises a 52×52 pixel frame.

18. The method of claim 17, wherein each 2D image sequence comprises 32 frames.

19. The method of claim 15, wherein the converting comprises tubelet embedding of the 2D image sequence and the first plurality of 2D image sequences.

20. The method of claim 19, wherein the tubelet embedding extracts non-overlapping spatiotemporal patches across the 2D image sequences.

21. The method of claim 20, wherein each patch comprises an 8×8 pixel frame.

22. The method of claim 21, wherein the converting comprises projecting the non-overlapping spatiotemporal patches as tokens into a vector.

23. The method of claim 12, wherein the video vision transformer model comprises a multi-head attention layer.

24. The method of claim 12, wherein the video vision transformer model comprises a feed forward neural network layer.

25. The method of claim 12, wherein output of the multi-head attention layer is fed into the feed forward neural network layer.
